(19) United States
(12) Patent Application Publication
(10) Pub. No.: US 2011/0189271 A1
(43) Pub. Date: Aug. 4, 2011

(54) PHARMACEUTICAL FORMULATIONS OF ACID-LABILE DRUGS

(76) Inventors: Vishal Lad, Hyderabad (IN); Ajmal MD Shareef, Hyderabad (IN); Vinay Muley, Hyderabad (IN); Sushant Dube, Hyderabad (IN); Ashwini Rewatkar, Hyderabad (IN); Rahul Sudhakar Gawande, Hyderabad (IN); Srinivas Irukulla, Hyderabad (IN); Surakumar Jayanthi, Hyderabad (IN)

(21) Appl. No.: 13/018,483

(22) Filed: Feb. 1, 2011

Related U.S. Application Data
(60) Provisional application No. 61/349,061, filed on May 27, 2010.

(30) Foreign Application Priority Data
Feb. 2, 2010 (IN) 235/CHE/2010

(51) Int. Cl. A61K 31/4439; A61K 9/00; A61K 9/20; A61K 9/14; A61K 9/48; A61P 1/00; A61P 1/04; A61K 9/24

(52) U.S. Cl. 424/451; 424/400; 514/338; 424/475; 424/490; 427/2.21

(57) ABSTRACT

Pharmaceutical formulations comprising inert cores that are coated with a layer comprising an amorphous dexlansoprazole sodium salt formed in-situ, further sequentially coated with an intermediate layer and an enteric coating layer. Coated cores may be further formulated to produce tablets or capsules.

### INTRODUCTION

Aspects of the present application relate to pharmaceutical formulations comprising at least one acid labile pharmaceutically active substance, or any salts, polymorphs, hydrates, esters, isomers, derivatives, or mixtures thereof, for oral use. Aspects of the present application also relate to processes for manufacturing such compositions and methods of preventing or treating diseases or disorders using such compositions. An aspect of the application provides methods for producing a gastric acid secretion inhibitory effect to a subject in need thereof, by administering an effective amount of the pharmaceutical compositions. Embodiments of the present application comprise at least one substituted benzimidazole derivative, such as dexlansoprazole, as the active agent. In embodiments, the compositions of the present application are highly stable and may comprise at least one basic compound, such as an alkaline metal compound, in order to enhance storage stability.

Substituted benzimidazoles are potent inhibitors of gastric acid secretion. These compounds are susceptible to degradation and/or transformations in both acid and neutral media. The acidic decomposition of these acid labile compounds is due to an acid catalyzed reaction. Therefore, such acid-labile drugs need to be formulated in a way to stabilize the compositions. These acid-labile drugs include substituted benzimidazole gastric anti-secretary agents, such as dexlansoprazole and lansoprazole and pharmaceutically acceptable salts thereof. These agents are known as proton pump inhibitors with powerful inhibitory action against secretion of gastric acid. They are indicated for the treatment of various digestive ulcers and are well known in the art.

When these acid-labile compounds are formulated into pharmaceutical preparations for oral administration, they require special techniques to avoid contact of drug with gastric acid of the stomach. One technique most commonly used is to coat the acid-labile compound, whether in granule, pellet or tablet form, with an enteric polymer coating, which is insoluble in water under acidic conditions and soluble in water under neutral to alkaline conditions. However, the material used in enteric coatings itself is acidic, which can cause the decomposition of the acid-labile compound. Such decomposition occurs even during the enteric coating process, which results in the discoloration of the surface of the drug-containing core. In order to avoid such problems, an inert sub/barrier coating, which is not acidic, is often required between the core and enteric coating, which increases the complexity and the cost of the formulation manufacturing processes involving acid-labile compounds.

The benzimidazole compound having an adopted name “dexlansoprazole” is an optical isomer of lansoprazole and has a chemical name (+)-2-[(R)-{[3-methyl-4-(2,2,2-trifluoroethoxy)pyridin-2-yl]nethyl}sulfinyl]-1H-benzimidazole, with structural Formula I.

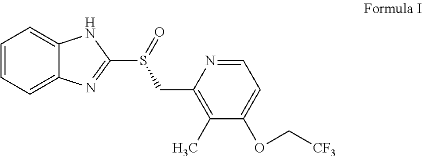

Dexlansoprazole is the active ingredient in DEXILANT™ delayed release capsules, sold by Takeda Pharmaceuticals America, Inc., and is useful for the prevention and treatment of gastric acid related disorders in mammals and man, including, e.g., gastroesophageal reflux disease, gastritis, gastric ulcers, duodenal ulcers, etc. Dexlansoprazole is susceptible to degradation/transformations in acid reacting and neutral media. The stability of dexlansoprazole is also affected by moisture, heat, and organic solvents, and to some degree by light.

U.S. Pat. Nos. 6,462,058 and 6,664,276 disclose crystalline forms of dexlansoprazole or a salt thereof. U.S. Pat. Nos. 4,628,098, 4,786,505, 4,853,230, 5,689,333, 5,045,321, 5,093,132, and 5,433,959 teach various stabilizing agents for their disclosed benzimidazole derivatives in core tablets. These patents also show that such compounds are stable in the presence of basic inorganic salts of magnesium, calcium, potassium and sodium. The stability is further enhanced by separating the acid-labile benzimidazoles from the acidic components of the enteric coating by interposing an intermediate coating (subcoating). U.S. Pat. No. 6,939,971 discloses a method of treating Zollinger-Ellison syndrome, reflux esophagitis and Helicobacter pylori infection by administering compositions containing crystalline dexlansoprazole.

U.S. Pat. No. 6,013,281, of which the entire content is incorporated herein by reference, discloses that a separating layer can be formed in situ by direct application of an acidic enteric material onto an alkaline core containing benzimidazoles. U.S. Patent Application Publication No. 2006/0057195A1 describes solid preparations for medicinal use containing amorphous benzimidazole compounds including dexlansoprazole, which are produced by blending an amorphous benzimidazole compound with a nontoxic base such as a basic inorganic salt. U.S. Pat. No. 7,790,755 describes capsules comprising: (i) a tablet, granule, or fine granule comprising a core particle containing the active ingredient and a pH dependently soluble release-controlled coating layer that releases the active in a pH range of 6 to 7.5; and (ii) a tablet, granule or fine granule comprising a core particle containing the active ingredient and an enteric coating, such that the active ingredient is released in the pH range of no less than 5 to no more than 6. U.S. Patent Application Publication No. 2007/0141137 describes capsules comprising a moisture-sensitive compound which is stable in a low moisture state and has pH-independent disintegration properties.

There remains a need for improved pharmaceutical compositions comprising dexlansoprazole.

### SUMMARY

Aspects of the present application relate to pharmaceutical compositions comprising at least one acid labile pharmaceutically active substance, or any salts, polymorphs, hydrates, esters, isomers, derivatives, or mixtures thereof, for oral administration, and processes for their preparation.

An aspect of the present application relates to modified release formulations comprising at least one substituted benzimidazole derivative, such as lansoprazole or a single enantiomer, for oral administration, and processes of their preparation.

In embodiments, the present application provides compositions comprising dexlansoprazole, together with one or more pharmaceutically acceptable excipients.

In embodiments, dexlansoprazole compositions of the present application are in the form of particles. In further embodiments, particles according to the present application may be in the form of powders, granules, pellets, spheroids, extrudates, mini-tablets, and the like.

In embodiments, dexlansoprazole compositions of the present application are in the form of particles made into a unit dosage form such as tablets or capsules.

In embodiments, dexlansoprazole compositions of the present application are in the form of powders or granules or pellets, compressed into tablets or filled into capsules.

In embodiments, compositions of the present application comprise dexlansoprazole in an amorphous form, crystalline form, or mixtures thereof as the active agent, together with at least one metal salt compound, and together with one or more other excipients.

In embodiments, dexlansoprazole compositions of the present application comprise: (a) a pharmacologically inert core; (b) a drug layer over the core comprising an amorphous dexlansoprazole metal salt, and one or more pharmaceutically acceptable excipients; (c) an intermediate layer over the drug layer; and (d) an enteric layer over the intermediate layer.

In embodiments, dexlansoprazole compositions of the present application comprise dexlansoprazole in an amorphous form as the active agent, together with at least one metal compound such as an alkaline metal compound and one or more other excipients, wherein the dexlansoprazole is at least partially converted into a dexlansoprazole salt of the alkaline metal in situ during the manufacturing process.

In embodiments, the present application provides dexlansoprazole compositions, prepared using dexlansoprazole, its salt, or mixtures thereof, having particle size distributions with D₅₀ about 1 μm to about 500 μm.

In embodiments, the present application provides dexlansoprazole compositions, prepared using dexlansoprazole, its salt, or mixtures thereof, having particle size distributions with D₅₀ about 1 μm to about 500 μm, and D₉₀ about 1 μm to about 1000 μm.

In embodiments, the present application provides compositions comprising dexlansoprazole or its pharmaceutically acceptable salts, which are substantially free of drug degradation impurities.

In embodiments, formulations of the present application may contain any one or more of impurities A, B, C, D, E, or any other drug-related impurity, in amounts such that any such impurities do not substantially adversely affect the safety of the composition.

In embodiments, the present application provides compositions comprising dexlansoprazole or its pharmaceutically acceptable salts, wherein levels of one or more of impurities A, B, C, D, or E, as described herein, are less than about 5%, or less than about 1%, of the label dexlansoprazole content.

In embodiments, the present application relates to compositions and/or formulations wherein levels of an adduct impurity are less than about 5%, or less than about 1%, of the label dexlansoprazole content.

In embodiments, the present application relates to stable compositions wherein total drug-related impurities, as determined using high performance liquid chromatograph (HPLC), are less than about 7%, or less than about 3%, of the label dexlansoprazole content.

In an aspect, the present application provides processes for preparing compositions of dexlansoprazole, embodiments of the process comprising: (a) combining amorphous dexlansoprazole, at least one metal compound, and one or more pharmaceutically acceptable excipients with a solvent to obtain a dexlansoprazole solution; (b) applying the dexlansoprazole solution obtained in (a) onto pharmacologically inert cores; (c) applying an intermediate layer onto the drug coated cores obtained in (b); and (d) applying an enteric coating layer onto the intermediate layer coated cores obtained in (c); wherein a dexlansoprazole metal salt is formed in situ during the process.

In embodiments, processes further comprise: (e) mixing enteric coated multiple unit cores with one or more pharmaceutically acceptable excipients; and (f) making the composition thus obtained into a unit dosage form such as a tablet or capsule.

In embodiments, the present application provides processes for producing drug-containing granules or pellets, wherein granules or pellets are processed in an environment where the relative humidity (RH) is not more than about 70%.

In embodiments of the present application, volume ratios of water to organic solvent in the drug solution for coating particles is about 100:0 to about 0:100.

In embodiments, the present application provides processes for producing drug-containing granules or pellets, including drying granules or pellets at temperatures of 50±20° C.

In embodiments, the present application provides processes for producing drug-containing granules or pellets, wherein granules or pellets contain water in amounts about 0.5-10% by weight, as determined using a Karl Fischer method.

In embodiments, the application provides processes for producing drug-containing granules or pellets, wherein granules or pellets have loss on drying in the range of about 0.25-10%, or about 0.5-5% by weight.

Aspects of the present application provides methods of preventing or treating diseases or disorders using compositions of the present application comprising at least one substituted benzimidazole derivative or its salts, or mixtures thereof.

Embodiments of the present application provide methods for producing a gastric acid secretion inhibitory effect in a subject in need thereof, by administering an effective amount of a pharmaceutical composition comprising amorphous dexlansoprazole or its salts or mixtures thereof.

In an aspect, compositions of the present application exhibit appreciable chemical stability during storage.

In embodiments, the present application provides multiple unit dosage forms, comprising: (a) a pharmacologically inert core; (b) a drug layer over the core comprising dexlansoprazole or its salt, or mixtures thereof, and one or more pharmaceutically acceptable excipients; (c) an intermediate layer over the drug layer; and (d) an enteric layer over the intermediate layer.

In embodiments, the present application provides multiple unit dosage forms, comprising: (a) a pharmacologically inert core; (b) a drug layer over the core comprising a dexlansoprazole sodium or potassium salt, and one or more pharmaceutically acceptable excipients; (c) an intermediate layer over the drug layer; and (d) an enteric layer over the intermediate layer.

In aspects, the present application provides processes for preparing dexlansoprazole compositions, embodiments comprising: (a) combining amorphous dexlansoprazole, sodium hydroxide, and one or more pharmaceutically acceptable excipients with a solvent to obtain a dexlansoprazole solution; (b) applying the dexlansoprazole solution obtained in (a) onto pharmacologically inert cores; (c) applying an intermediate layer onto the drug coated cores obtained in (b); and (d) applying an enteric coating layer onto the intermediate layer coated cores obtained in (c); wherein a dexlansoprazole metal salt is formed in situ during the process.

In embodiments, the active agent dexlansoprazole is contained in compositions as a free base or salt form during manufacture or storage, in an amorphous form, or crystalline form, or mixtures thereof.

### BRIEF DESCRIPTION OF THE DRAWINGS

FIG. 1 is a X-ray powder diffraction (XRPD) pattern of a dexlansoprazole formulation prepared according to Example 1.

FIG. 2 is a XRPD pattern of a dexlansoprazole formulation prepared according to Example 1, after storage for 3 months at 40° C. and 75% RH.

### DETAILED DESCRIPTION

All percentages and ratios used herein are by weight of the total composition and all measurements made are at 25° C. and normal pressure unless otherwise designated. All temperatures are in degrees Celsius unless specified otherwise. As used herein, “comprising” means the elements recited, or their equivalent in structure or function, plus any other element or elements that are not recited. The terms “having” and “including” are also to be construed as open ended unless the context suggests otherwise. As used herein, “consisting essentially of” means that the application may include ingredients in addition to those recited in the claim, but only if the additional ingredients do not materially alter the basic and novel characteristics of the claimed application. All ranges recited herein include the endpoints, including those that recite a range “between” two values. The terms “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error, and instrument error for a given technique used to measure a value.

Where this document refers to a material, such as, for example, for instance, a benzimidazole drug, and the unique solid and/or crystalline or amorphous forms, salts, solvates, and/or optical isomers thereof, by reference to patterns, spectra, or other graphical data, it may do so by qualifying that they are “substantially” as shown or depicted in a figure, or by one or more data points. It will be appreciated that patterns, spectra, and other graphical data may be shifted somewhat in their positions, relative intensities, or other values due to a number of factors known to those of skill in the art. For example, in the crystallographic and powder X ray diffraction arts, shifts in peak positions, or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art will be able to compare the figures herein with a pattern generated of an unknown form of, for instance, a benzimidazole drug, and confirm its identity as one of the forms disclosed and claimed herein. The same holds true for other techniques which may be reported herein, as well as for distinguishing between amorphous forms.

In addition, where a reference is made to a drawing, it is permissible to select any number of data points illustrated in the drawing that uniquely define that crystalline or amorphous form, salt, solvate, and/or optical isomer, within any associated and recited margin of error, for purposes of identification.

Unless specified otherwise, the word “pure” as used herein means that the material has at least about 99% purity. In general, this refers to purity with regard to unwanted residual solvents, reaction by-products, impurities, and unreacted starting materials. In the case of stereoisomers, “pure” as used herein also means 99% of one enantiomer or diastereomer, as appropriate. “Substantially pure” as used herein means at least about 98% purity and, likewise, “essentially pure” as used herein means at least about 95% purity.

The phrase “substantially free of one or more of its corresponding impurities” as used herein, unless otherwise defined, means comprising less than about 7%, or less than about 5%, or less than about 3%, or less than about 2%, or less than about 1%, or less than about 0.5%, or less than about 0.3%, or less than about 0.1%, or less than about 0.05% by weight, of one or more of the impurities, as measured by high performance liquid chromatography (HPLC) or other analytical techniques.

The term “benzimidazole compound” as used herein refers to any of the compounds belonging to the category of substituted benzimidazoles used for treating gastrointestinal disorders, including esomeprazole, lansoprazole, rabeprazole, pantoprazole, leminoprazole and pariprazole, including their single enantiomers and pharmaceutically acceptable salts, solvates, and mixtures. For example, the benzimidazole compound may be dexlansoprazole in the form of the free base, or a pharmaceutically acceptable salt thereof, in any polymorphic form.

Like other substituted benzimidazole derivatives, dexlansoprazole is acid-labile, creating several problems in formulating into oral pharmaceutical dosage forms because of the acidic environment of the stomach. It has poor stability and is rapidly decomposed and discolored under moist conditions, or in an acidic to neutral aqueous environment. It requires special techniques to avoid contact of the drug with gastric acid of the stomach. Even though stabilization of substituted benzimidazole derivatives has been described previously, there remains a need for alternate approaches to preparing stable and bioavailable pharmaceutical compositions comprising dexlansoprazole.

As used herein the term “dexlansoprazole” includes the compound dexlansoprazole, pharmaceutically acceptable salts, esters, prodrugs thereof, the active metabolites of dexlansoprazole and the prodrugs thereof, and their polymorphs, solvates, and hydrates.

The terms “pharmaceutically acceptable salt” as used herein refers to salts which are known to be non-toxic and are commonly used in pharmaceutical practice. Such pharmaceutically acceptable salts include metal salts, salts with organic bases, salts with basic amino acids, etc. Metal salts include, for example, alkali metal salts, such as sodium salt and potassium salts, and alkaline earth metal salts, such as calcium, magnesium and barium salts. Salts with organic bases include, for example, salts with trimethylamine, triethylamine, pyridine, picoline, ethanolamine, diethanolamine, triethanolamine, dicyclohexylamine, N,N-dibenzylethylenediamine, etc. Salts with basic amino acids include, for example, salts with arginine, lysine, etc. Acid addition salts such as hydrochloride salts and the like are also included.

The term “acid-labile compound” means a compound that is not stable in acidic environments, or which undergoes degradation or hydrolysis via acid or proton catalyzed reactions.

The term “excipient” means a component of a pharmaceutical product that is not an active ingredient, such as a filler, diluent, carrier, etc. The excipients that are useful in preparing a pharmaceutical composition are generally safe, non-toxic and neither biologically nor otherwise undesirable, and are acceptable for veterinary use as well as human pharmaceutical use. Reference to an excipient includes both one and more than one such excipient.

The term “formulation” refers to an oral dosage form such as a tablet or capsule, comprising a benzimidazole drug.

The term “intermediate layer” refers to A barrier layer or subcoating, formed between a drug layer and an enteric layer.

In an aspect, the present application provides solid dosage forms comprising amorphous benzimidazole compounds blended with a non-toxic base, such as a basic inorganic salt, in a core particle, and further comprising a subcoating or barrier coating and an enteric coating layer on the drug containing core particle. Processes for the manufacture of such dosage forms represent further aspects of the present application.

A core may be in the form of pellets, granules, beads, etc. The core may be acidic, alkaline, or neutral, depending on the type of formulation. The core may contain one or more pharmaceutically acceptable excipients, such as inert carriers, binders, diluents, disintegrants, lubricants/glidants, solubilizers/wetting agents, and any mixtures thereof. A core may be coated with a benzimidazole compound and one or more of binders, diluents, disintegrants, lubricants/glidants, solubilizers/wetting agents, and mixtures thereof. The core may comprise substances such as starch, microcrystalline cellulose, or sugar spheres, such as nonpareil sugar seeds.

In embodiments, the benzimidazole compound is dexlansoprazole. In embodiments, dexlansoprazole formulations of the present application are in the form of multi-particulates. In further embodiments, the multi-particulates may be in the form of powders, granules, pellets, spheroids, mini-tablets, and the like.

In embodiments, dexlansoprazole formulations of the present application are in the form of multi-particulates made into a unit dosage form such as capsules.

In embodiments, dexlansoprazole formulations of the present application are in the form of pellets or mini-tablets filled into capsules. In embodiments, dexlansoprazole formulations of the present application comprise a single fraction of multi-particulates, such as pellets or mini-tablets, filled into capsules, wherein the multi-particulate fraction comprises cores containing the drug for extended release, followed by a layer of drug coating for immediate release. These are further coated with an enteric polymer, wherein the multi-particulates are optionally coated to form a subcoating layer prior to enteric coating.

In embodiments, dexlansoprazole formulation of the present application comprise at least two fractions of multi-particulates, such as mini-tablets or pellets, filled into capsules, wherein one fraction of multi-particulates is coated with an enteric polymer dissolving or decomposing in a pH range between about 3 and 7 to release the active agent, and wherein another fraction of multi-particulates is coated with an enteric polymer dissolving or decomposing in a pH range between about 4 and 8 to release the active agent, and wherein the multi-particulates in each of the two fractions are optionally coated to form a subcoating layer prior to enteric coating.

In embodiments, the present application comprises delayed release solid oral dosage forms comprising dexlansoprazole or pharmaceutically acceptable salts thereof, wherein the dosage forms release less than about 20% of active ingredient within about the first 120 minutes after immersion into 750 or 1000 mL of a 0.1N hydrochloric acid (pH 1.2) dissolution medium, using test method 711 “Dissolution” in United States Pharmacopeia 29, United States Pharmacopeial Convention, Inc., 2005 (“USP”), and type 2 apparatus. The release of the drug occurs thereafter, in the range of about 10% to about 45% of drug dissolving within about 90 minutes and about 50% to about 100% of the drug dissolving within about 300 minutes.

In embodiments, pharmaceutical compositions comprising dexlansoprazole further comprise a pharmaceutically acceptable organic base. Organic bases that may be used in the present application are pharmaceutically acceptable organic bases, including, for example, meglumine, lysine, N,N′-dibenzylethylenediamine, chloroprocain, choline, diethanolamine, ethylenediamine, procaine, and mixtures thereof.

Organic solvents that may be used in processes of the present application include, but are not limited to: halogenated hydrocarbons, such as dichloromethane, 1,2-dichloroethane, chloroform and carbon tetrachloride; alcohols, such as methanol, ethanol, isopropyl alcohol, 1-propanol, 2-propanol, 1-butanol, 2-butanol, and t-butyl alcohol; ketones, such as acetone, ethyl methyl ketone, diethyl ketone, and methyl isobutyl ketone; esters, such as ethyl acetate, n-propyl acetate, n-butyl acetate and t-butyl acetate; ethers, such as diethyl ether, dimethyl ether, diisopropyl ether, methyl t-butyl ether and 1,4-dioxane; nitriles, such as acetonitrile and propionitrile; and any mixtures thereof.

Various stabilizers for use in formulations of the application to reduce the degradation of dexlansoprazole during storage include organic and inorganic bases and alkaline substances. Various useful basic inorganic compounds include, but are not limited to, sodium, potassium, calcium, magnesium, and aluminum compounds. Examples of sodium compounds are sodium carbonate, sodium hydrogen carbonate, sodium hydroxide, and the like. Examples of potassium compounds are potassium carbonate, potassium hydrogen carbonate, potassium hydroxide, and the like. Examples of magnesium compounds are heavy magnesium carbonate, magnesium carbonate, magnesium oxide, magnesium hydroxide, magnesium metasilicate aluminate, magnesium silicate, magnesium aluminate, synthetic hydrotalcite [Mg₆Al₂(OH)₁₆.CO₃.4H₂O], aluminum hydroxide-magnesium [2.5MgO.Al₂O₃.xH₂O], and the like. Examples of calcium compounds include precipitated calcium carbonate, calcium hydroxide, and the like. Organic bases that may be used in the present application are pharmaceutically acceptable organic bases, including, for example, meglumine, lysine, N,N′-dibenzylethylenediamine, chloroprocain, choline, diethanolamine, ethylenediamine, procaine, and mixtures of any two or more thereof.

In embodiments, an alkaline metal compound used in the compositions of the present application is one or more sodium salts that partially convert dexlansoprazole in situ into its corresponding sodium salt. In embodiments, an alkaline metal salt used in the compositions of the present application is one or more sodium salts which completely convert dexlansoprazole in situ into its corresponding sodium salt. In embodiments, an alkaline metal salt used in the compositions of the present application is one or more sodium salt which is present in an amount in excess of a stoichiometric amount required to convert the entire amount of dexlansoprazole into its corresponding sodium salt. In embodiments, an alkaline metal salt is useful in stabilizing the drug composition. In embodiments, an alkaline metal salt is present in amounts about 0.1% to about 50% by weight of the composition.

In embodiments, compositions of the present application comprise: (a) a pharmacologically inert core; (b) a drug layer over the core, comprising an amorphous dexlansoprazole metal salt and one or more pharmaceutically acceptable excipients; (c) an intermediate layer over the drug layer; and (d) an enteric layer over the intermediate layer.

In embodiments, compositions of the present application comprise dexlansoprazole in an amorphous form as the active agent, together with at least one metal compound, such as an alkaline metal compound, and one or more other excipients, wherein the dexlansoprazole is at least partially converted into a dexlansoprazole salt of the alkaline metal in situ during the manufacturing process.

In embodiments, an inert subcoating separates a core from an enteric coating polymer that contains free carboxyl groups, which may cause degradation and/or discoloration. The inert subcoating may also serve as a pH-buffering zone, in which hydrogen ions diffusing from the outside toward the alkaline core can react with hydroxyl ions diffusing from the alkaline core toward the surface of the coated articles. A subcoating may comprise one or more layers.

An inert subcoating can be applied to core pellets or mini-tablets, for example using conventional coating procedures in a suitable coating pan or in fluidized bed apparatus using water and/or conventional organic solvents for the coating solutions or dispersions. Water soluble or insoluble polymers that can be used for an inert subcoating include, for example, sugars, zein, hydroxypropyl celluloses, hydroxypropyl methylcelluloses, hydroxyethyl celluloses, polyvinyl alcohols, polyethylene glycols, poloxamers (Pluronic™ products), ethyl celluloses, gelatins, polyarginines, polyglycines, polyvinylpyrrolidones, vinyl acetate copolymers, and any mixtures thereof.

In embodiments, ane active agent dexlansoprazole is contained in compositions as a free base or salt form during manufacture or storage, in an amorphous form, a crystalline form, or mixtures thereof. The dexlansoprazole base may be present in an amorphous form or a crystalline form, or mixtures thereof. Dexlansoprazole sodium may be present in an amorphous form, a crystalline form, or mixtures thereof. In embodiments, compositions of the present application are substantially free of crystalline dexlansoprazole base and/or dexlansoprazole sodium.

According to the present application, a binder that is present may be any pharmaceutically acceptable, non-toxic pharmaceutically acceptable binder. In embodiments, the binder is a water soluble polymer such as a polyvinyl alcohol, a polyvinylpyrrolidone, a methylcellulose, a hydroxypropyl cellulose, a hydroxymethyl cellulose, a hydroxypropyl methylcellulose, and the like, including any mixtures thereof.

An enteric coating can be applied either directly onto cores or onto subcoated cores using, for example, conventional coating techniques such as, for instance, pan coating or fluidized bed coating using solutions of pH dependent polymers in water and/or suitable organic solvents, or by using suspensions of polymers, to provide a modified release of the active agent. Generally, an enteric coating is applied in amounts about 5% to about 100% by weight of the uncoated cores or sub-coated cores.

Enteric coating polymers that can be used include, for example, cellulose acetate phthalates (CAP), hydroxypropyl methylcellulose phthalates (HPMCP), polyvinyl acetate phthalates (PVAP), hydroxypropyl methylcellulose acetate succinates (HPMCAS), cellulose acetate trimellitates, hydroxypropyl methylcellulose succinates, cellulose acetate succinates, cellulose acetate hexahydrophthalates, cellulose propionate phthalates, copolymers of methylmethacrylic acid and methyl methacrylate, copolymers of methyl acrylate, methylmethacrylate and methacrylic acid, copolymers of methylvinyl ether and maleic anhydride (Gantrez™ ES products), ethyl methyacrylate-methyl methacrylate-chlorotrimethylammonium ethyl acrylate copolymers, natural resins such as zein, shellac, and copal collophorium, carboxymethyl ethylcelluloses, co-polymerized methacrylic acid/methacrylic acid methyl esters such as, for instance, materials sold under the trade name Eudragit® L12.5, L100, or Eudragit® S12.5, S100, and several commercially available enteric dispersion systems (e.g., Eudragit® L30D55, Eudragit® FS30D, Eudragit® L100-55, Eudragit® S100 (Rohm Pharma), Kollicoat® MAE30D and 30DP (BASF), Estacryl® 30D (Eastman Chemical), Aquateric® and Aquacoat® CPD30 (FMC), and any mixtures thereof.

Typical solvents that may be used to apply enteric coatings include isopropyl alcohol, acetone, methylene chloride, isopropyl alcohol with water, and the like, and any mixtures thereof.

Enteric coatings of the present application may comprise a plasticizer present in an amount of 0% to about 50% by weight of the enteric coating composition. Suitable plasticizers include acetyl triethyl citrate, dibutyl phthalate, tributyl citrate, triethyl citrate, acetyl tributyl citrate, propylene glycol, triacetin, polyethylene glycol, and diethyl phthalate. The enteric coating layer may further comprise a dispersant such as talc. Colorants and pigments may also be included in the enteric coating layer.

Coating materials of the present application may comprise lubricants such as calcium stearate, magnesium stearate, stearic acid, syloid, a coagulated aerosol of synthetic silica, a pyrogenic silicon dioxide, etc.

Compositions of the application can be processed into various pharmaceutical dosage forms as prepared, or can be combined with one or more pharmaceutically acceptable excipients. The different pharmaceutical dosage forms which comprise pharmaceutical compositions of the present application include solid oral dosage forms such as, but not limited to, powders, granules, pellets, tablets, and capsules.

Modified release compositions may comprise hydrophilic, lipophilic, or hydrophobic release rate controlling substances, or their combinations, to form a matrix or reservoir, or combinations of matrix and reservoir systems. The compositions may be prepared using any techniques, including direct blending, dry granulation, wet granulation (aqueous or non-aqueous, or partly aqueous and partly non-aqueous or aqueous-alcoholic), and extrusion and spheronization. Compositions may be presented as uncoated, film coated, sugar coated, compression-coated, powder coated, enteric coated, or modified release coated forms.

One or more polymers that can be used in present application for modified release include hydrophilic, hydrophobic, and lipophilic substances, and combinations thereof. Examples of suitable polymers include, without limitation thereto, cellulose ethers, e.g., hydroxypropyl methylcelluloses (hypromellose or HPMC), hydroxypropylcelluloses (HPC), hydroxyethylcelluloses, ethylcelluloses, and carboxymethylcellulose sodium, polyvinylpyrrolidones, including non-crosslinked polyvinylpyrrolidones, carboxymethylstarches, polyethylene glycols, polyoxyethylenes, poloxamers (polyoxyethylene-polyoxypropylene copolymers), polyvinyl alcohols, glucanes (glucans), carrageenans, scleroglucanes (scleroglucans), mannans, galactomannans, gellans, alginic acid and derivatives thereof (e.g., sodium or calcium alginate, propylene glycol alginate), polyaminoacids (e.g., gelatin), methylvinyl ether/maleic anhydride copolymers, polysaccharides (e.g. carageenan, guar gum, xanthan gum, tragacanth and ceratonia), alpha-, beta- or gamma-cyclodextrins, dextrin derivatives (e.g. dextrin), polymethacrylates (e.g. copolymers of acrylic and methacrylic acid esters containing quaternary ammonium groups), acrylic acid polymers (e.g., carbomers), shellac and derivatives thereof, cellulose acetate, cellulose butyrate, cellulose diacetate, cellulose triacetate, cellulose propionate, cellulose acetate butyrate and other acetylated cellulose derivatives, and the like, and mixtures thereof.

Examples of lipophilic/hydrophobic substances that can be used in the present application include, without limitation thereto, waxes (e.g., carnauba wax, microcrystalline wax, beeswax, and polyethoxylated beeswax), natural fats (coconut, soya, cocoa) including modified forms such as totally or partially hydrogenated, hydrogenated castor oil, hydrogenated vegetable oil, and fatty acid derivatives such as mono-, bi- and tri-substituted glycerides, phospholipids, glycerophospholipids, glyceryl palmitostearate, glyceryl behenate, glyceryl monostearate, diethyleneglycol palmitostearate, polyethyleneglycol stearate, polyethyleneglycol palmitostearate, polyoxyethylene-glycol palmitostearate, glyceryl monopalmitostearate, cetyl palmitate, fatty alcohols associated with polyethoxylate fatty alcohols, cetyl alcohol, stearic acid, saturated or unsaturated fatty acids and their hydrogenated derivatives, lecithin, cephalins, chitosan and derivatives thereof, sphingolipids, sterols such as cholesterol and its substituted derivatives, and the like, and mixtures thereof.

In embodiments, dexlansoprazole is used for preparing inclusion complexes of drug with cyclodextrins. In further embodiments, an amorphous form of dexlansoprazole is used for preparing inclusion complexes with cyclodextrins.

As used herein, “cyclodextrin” refers to any of the natural cyclodextrins, α-cyclodextrin, β-cyclodextrin, and γ-cyclodextrin, and their respective derivatives or analogs. Cyclodextrins (sometimes called cycloamyloses) make up a family of cyclic oligosaccharides, composed of 5 or more α-D-glucopyranoside units linked 1→4, as in amylose (a fragment of starch). The formation of the inclusion compounds greatly modifies the physical and chemical properties of the guest molecules (such as dexlansoprazole in embodiments of the present application), mostly in terms of water/aqueous solubility. An inclusion complex of dexlansoprazole with cyclodextrins also aids in penetration of the drug into body tissues.

Any cyclodextrin which enhances the aqueous solubility and/or provides for effective delivery of dexlansoprazole may be used in the present application. The cyclodextrins of the present application can include the natural occurring cyclodextrins and their derivatives. The natural cyclodextrins include α-cyclodextrin, β-cyclodextrin and γ-cyclodextrin. Derivatives are typically prepared by modifying the hydroxyl groups located on the exterior or hydrophilic side of the cyclodextrin. The modifications can be made to increase the aqueous solubility and the stability of the complexes and can modify the physical characteristics of the complexes, including the formation and dissociation of the complex. The types and degrees of modification, as well as their preparation, are well-known in the art.

Any of the natural cyclodextrins can be derivatized, such as derivatives of β-cyclodextrin. Cyclodextrin derivatives include alkylated cyclodextrins, comprising methyl-, dimethyl-, trimethyl- and ethyl-β-cyclodextrins; hydroxyalkylated cyclodextrins, including hydroxyethyl-, hydroxypropyl-, and dihydroxypropyl-β-cyclodextrin; ethylcarboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin.

Other cyclodextrins include the naturally occurring cyclodextrins, methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, 3-hydroxypropyl-β-cyclodextrin, β-cyclodextrin sulfate, β-cyclodextrin sulfonate, or β-cyclodextrin sulfobutyl ether. Any of the above cyclodextrins or their derivatives or polymers prepared from them can be used for preparation of the compositions of the application, either alone or in the form of mixtures of one or more cyclodextrins.

In embodiments, pharmaceutical compositions of the present application comprise dexlansoprazole, adsorbed onto at least one pharmaceutically acceptable carrier. Carriers according to the present application include, but are not limited to, polyvinylpyrrolidones, hydroxypropyl methylcelluloses, sugar substances such as mannitol, sorbitol, etc. and the like.

In embodiments, the present application includes modified release pharmaceutical compositions comprising dexlansoprazole, optionally together with one or more pharmaceutically acceptable excipients, wherein said compositions are in multi-particulate form.

In embodiments, the present application includes modified release pharmaceutical compositions comprising cores containing dexlansoprazole, optionally together with one or more pharmaceutically acceptable excipients, and a coating comprising one or more polymers, wherein the compositions are in multi-particulate form.

In embodiments, modified release multi-particulates comprise non-pariel cores, such as inert sugar or similar substances, upon which dexlansoprazole is loaded, optionally together with one or more pharmaceutically acceptable excipients, using any suitable technique, such as powder layering, solution spraying, suspension spraying, or any other techniques.

In embodiments, modified release compositions of the application comprise dexlansoprazole loaded non-pariel cores having a coating comprising one or more pH independent polymers, pH dependent polymers, or combinations thereof.

In embodiments, the present application includes pharmaceutical compositions comprising modified release multi-particulates comprising dexlansoprazole-containing cores and a coating comprising one or more polymers, and optionally having one or more further coatings.

In embodiments, multi-particulates comprising dexlansoprazole further contain one or more non-functional coatings or functional coatings, to provide modified release of the active agent.

Multi-particulate formulations of the application can be prepared using the techniques described herein, as well as other methods known to those having skill in the art.

In embodiments, multi-particulates comprising dexlansoprazole are coated with different concentrations of polymers, giving portions having different release profiles, and these can be combined to form a pharmaceutical composition or dosage form to achieve desired modified release profiles.

In embodiments, multi-particulates comprising dexlansoprazole are coated with different types of polymers, either enteric polymers (pH dependent polymers) or modified release polymers (pH independent polymers), giving different release profiles, and these can be combined to form a pharmaceutical composition or dosage form to achieve desired modified release profiles.

In embodiments, multi-particulates comprising dexlansoprazole can be combined with pharmaceutically acceptable excipients and compounded to form a pharmaceutical composition, which can be compressed into tablets or placed into suitable capsule shells using techniques known to those having skill in the art. In embodiments, the compositions of the present application are made into a hard gelatin capsule, wherein the empty hard gelatin capsule shells comprise one or more of hydroxymethyl cellulose, carrageenan, potassium chloride, polyvinyl polymers such as polyvinyl acetate and polyvinyl alcohol, and the like.

Useful pharmaceutical excipients according to the present application include, for example, any one or more of diluents, binders, stabilizers, lubricants, glidants, disintegrating agents, anti-oxidants, surfactants, and other additives that are commonly used in solid pharmaceutical dosage form preparations.

Various useful fillers or diluents according to the present application include, but are not limited to, starches, lactose, mannitol (e.g., Pearlitol™ SD200), cellulose derivatives, confectioner's sugar and the like. Different grades of lactose include, but are not limited to, lactose monohydrate, lactose DT (direct tableting), lactose anhydrous, Flowlac™ (available from Meggle Products), Pharmatose™ (available from DMV), and others. Different starches include, but are not limited to, maize starch, potato starch, rice starch, wheat starch, pregelatinized starch (commercially available as PCS PC10 from Signet Chemical Corporation), starch 1500, starch 1500 LM grade (low moisture content grade) from Colorcon, fully pregelatinized starch (commercially available as National 78-1551 from Essex Grain Products), and others. Different cellulose compounds that can be used include crystalline cellulose and powdered cellulose. Examples of crystalline cellulose products include, but are not limited to, Ceolus™ KG801, Avicel™ PH101, PH102, PH301, PH302 and PH-F20, PH-112 microcrystalline cellulose 114, and microcrystalline cellulose 112. Other useful diluents include, but are not limited to, carmellose, sugar alcohols such as mannitol (e.g., Pearlitol™ SD200), sorbitol, and xylitol, calcium carbonate, magnesium carbonate, dibasic calcium phosphate, and tribasic calcium phosphate.

Various useful binders according to the present application include, but are not limited to, hydroxypropylcelluloses, also called HPC (e.g., Klucel™ LF, Klucel™ EXF) and useful in various grades, hydroxypropyl methylcelluloses, also called hypromellose or HPMC (e.g., Methocel™ products) and useful in various grades, polyvinylpyrrolidones or povidones (such as grades PVP-K25, PVP-K29, PVP-K30, and PVP-K90), copovidones (e.g., Plasdone™ S 630), powdered acacia, gelatin, guar gum, carbomers (e.g., Carbopol® products), methylcelluloses, polymethacrylates, and starches.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, including but not limited, to crosslinked povidone, Kollidon™ CL from BASF (Germany), Polyplasdone™ XL, XI-10, and INF-10 from ISP Inc. (USA), and low-substituted hydroxypropylcelluloses. Examples of low-substituted hydroxypropylcelluloses include, but are not limited to, low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

Useful surface-active agents for the present application include non-ionic, cationic, anionic, and zwitterionic surface-active agents. Useful non-ionic surface-active agents include ethylene glycol stearates, propylene glycol stearates, diethylene glycol stearates, glycerol stearates, sorbitan esters (e.g., Span™ products) and polyhydroxyethylenically treated sorbitan esters (e.g., Tween™ products), aliphatic alcohols and PEG ethers, phenol and PEG ethers. Useful cationic surface-active agents include quaternary ammonium salts (e.g., cetyltrimethylammonium bromide) and amine salts (e.g., octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as, for example, phospholipids, e.g. diacylphosphatidyl glycerols, diaceylphosphatidyl cholines, diaceylphosphatidic acids, and the precursors and derivatives thereof, such as, for example, soybean lecithin, and egg yolk.

In embodiments, compositions of the present application contain at least one antioxidant. The antioxidant may be present either as a part of the composition or as a packaging component. Thus, in particular embodiments of the present application, antioxidants are introduced into the formulation during the drug loading stage, over inert cores. The antioxidants are present in amounts effective to retard decomposition of dexlansoprazole.

In embodiments, antioxidant contents of formulations range from about 0.001 to 10 weight percent, with respect to the active agent.

Among the antioxidants, non-limiting examples include ascorbic acid and its salts, sulfite salts such as sodium metabisulfite or sodium sulfite, sodium sulfide, tocopherols such as dl-alpha-tocopherol, butylated hydroxyanisole, butylated hydroxytoluene, ascorbyl palmitate, and propyl gallate. Other suitable antioxidants will be readily recognized by those skilled in the art.

Useful lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

One or more glidant materials, which improve the flow of powder blends, pellets and mini-tablets, and minimize dosage form weight variations, can be used. Useful glidants include, but are not limited to, silicon dioxide, talc, and combinations thereof.

Coloring agents can be used to color code the compositions, for example, to indicate the type and dosage of the therapeutic agent therein. Coloring agents can also be used to differentiate the varied fractions of multi-particulates comprising a unit dosage form such as a capsule. Suitable coloring agents include, without limitation, natural and/or artificial compounds such as FD&C coloring agents, natural juice concentrates, pigments such as titanium oxide, silicon dioxide, iron oxides and zinc oxide, and the like, and combinations thereof.

Various solvents can be used in processes of the present application, including, but not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and mixtures thereof.

pH-independent polymers according to the present application include, but are not limited to, carbomers, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidones, polyvinyl acetates, polyvinyl alcohols, polyglycolides, polysiloxanes, polyurethanes and copolymers thereof, alkyl celluloses, hydroxyalkyl celluloses, cellulose ethers, cellulose esters, nitrocelluloses, methyl celluloses, ethyl celluloses, hydroxypropyl celluloses, hydroxypropyl methylcelluloses, hydroxybutyl methyl celluloses, natural polymers such as alginates and other polysaccharides, including, but not limited to, arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starches and various other natural homopolymer or heteropolymers such as those containing one or more of aldoses, ketoses, acids or amines, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof, and including dextran and cellulose, collagen, chemical derivatives thereof (e.g., substitutions through additions of chemical groups, for example, alkyl, alkylene, hydroxylations, oxidations, and other modifications routinely made by those skilled in the art), albumin and other hydrophilic proteins, zein and other prolamines and hydrophobic proteins, synthetic polymers such as polymers of lactic acid and glycolic acid, polyanhydrides, poly(ortho esters), polyurethanes, poly(butyric acid), poly(valeric acid), poly(caprolactone), poly(hydroxybutyrate), poly(lactide-co-glycolide) and poly(lactide-co-caprolactone) copolymers, and mixtures thereof.

Various pH-dependent polymers for use in the present application include, but are not limited to, Eudragit® 100, Eudragit® RSPO and RLPO, Eudragit® ND 40, polymers and copolymers of acrylic and methacrylic acids, cellulose acetate butyrates, cellulose acetate phthalates, hydroxypropyl methylcellulose phthalates, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), and any mixtures thereof.

In embodiments, one or more pH independent or pH dependent polymers will be used for coating the compositions of the present application.

Other useful additives for coating include, but are not limited to, plasticizers, antiadherents, opacifiers, solvents, and optionally colorants, lubricants, pigments, antifoam agents, and polishing agents.

Various useful plasticizers include, but are not limited to, substances such as castor oil, diacetylated monoglycerides, dibutyl sebacate, diethyl phthalate, glycerin, polyethylene glycol, propylene glycol, triacetin, and triethyl citrate, and mixtures thereof. The type of plasticizer depends upon the type of coating agent. An opacifier like titanium dioxide may also be present in amounts ranging from about 0.5% to about 20%, based on the total weight of the coating.

Anti-adhesives are frequently used in film coating processes to avoid sticking effects during film formation and drying. An example of a useful anti-adhesive for this purpose is talc. The anti-adhesive is frequently present in the film coating in amounts about 0.5% (w/w) to 15% (w/w), based upon the total weight of the coating.

The foregoing descriptions of excipients are not intended to be exhaustive. Those skilled in the art will be aware of many other substances that are useful in the practice of the application, and the use of such substances is specifically included in this application.

In an aspect, the present application includes methods of preparing pharmaceutical compositions of the present application.

In embodiments, the present application includes stabilized pharmaceutical compositions of dexlansoprazole, which may be prepared by spray drying a suspension or solution of dexlansoprazole and a water soluble sugar derivative, with or without an organic base, optionally together with one or more pharmaceutically acceptable excipients. Alternatively, dexlansoprazole compositions may also be prepared by fluid bed granulation techniques, wherein a solution of dexlansoprazole, with or without a stabilizer, optionally together with one or more pharmaceutically acceptable excipients, is sprayed onto inert cores or layered onto inert cores.

In an aspect, the present application provides processes for producing stable compositions of dexlansoprazole, embodiments comprising: (a) combining amorphous dexlansoprazole, at least one metal compound, and one or more pharmaceutically acceptable excipients with a solvent to obtain a dexlansoprazole solution; (b) applying the dexlansoprazole solution obtained in (a) onto pharmacologically inert cores; (c) applying an intermediate layer onto the drug coated cores obtained in (b); and (d) applying an enteric coating layer onto the intermediate layer coated cores obtained in (c); wherein a dexlansoprazole metal salt is formed in situ during the process.

In specific embodiments, a composition of the present application may be prepared by a process including: (a) dissolving dexlansoprazole or a pharmaceutically acceptable salt thereof in an organic solvent; (b) optionally adding one or more pharmaceutically acceptable excipients such as a metal compound, stabilizer, a binder, polymer and/or a disintegrant to the solution; (c) spraying the solution onto a substrate comprising at least one diluent, optionally together with a disintegrant, to obtain a granulated mass; (d) drying the granules; (e) optionally, milling the granules; (f) mixing one or more excipients such as diluent, disintegrant, lubricant and/or glidant with the dried granules of (d) or (e); (g) compressing the material of (f) to form mini-tablets; (h) optionally subcoating the mini-tablets; (i) coating the mini-tablets of (g) or (h) with a pH dependent polymer or a pH independent polymer; and (j) filling the coated mini-tablets into a capsule.

In embodiments, a capsule contains at least two fractions of coated mini-tablets, wherein one fraction is coated with one or more pH independent polymers and another fraction is coated with one or more pH dependent polymers.

In embodiments, a capsule comprises at least two fractions of coated mini-tablets, wherein fractions are independently coated with one or more pH independent polymers or one or more pH dependent polymers.

In specific embodiments, a composition of the present application may be prepared by processes including: (a) seal coating a substrate; (b) dissolving a metal compound and dexlansoprazole, or a pharmaceutically acceptable salt thereof, in a solvent; (c) optionally, adding one or more pharmaceutically acceptable excipients such as a diluent, binder, polymer, and/or a disintegrant to the solution; (d) spraying the solution onto a seal coated substrate to obtain a granulated mass; (e) drying the granules; (f) optionally milling granules; (g) mixing one or more excipients, such as a diluent, disintegrant, lubricant, and/or glidant with the granules of (e) or (f); (h) compressing the mixture of (g) to form mini-tablets; (i) optionally, subcoating the mini-tablets; (j) coating the mini-tablets of (h) or (i) with a pH dependent polymer or a pH independent polymer; and (k) filling the coated mini-tablets thus obtained into capsules.

In embodiments, a capsule comprises at least two fractions of coated mini-tablets, wherein one fraction is coated with one or more pH independent polymers and another fraction is coated with one or more pH dependent polymers.

In embodiments, a capsule comprises at least two fractions of coated mini-tablets, wherein fractions are independently coated with one or more pH independent polymers or one or more pH dependent polymers.

In embodiments, dexlansoprazole compositions may be prepared using powder layering techniques, wherein a powder comprising dexlansoprazole, with or without a stabilizer, optionally together with one or more pharmaceutically acceptable excipients, is layered onto inert cores while spraying with a binder solution.

In specific embodiments, compositions of the present application may be prepared by processes including: (a) preparing a drug layering powder by mixing the drug and a diluent, optionally along with one or more pharmaceutically acceptable excipients such as a stabilizer and/or a disintegrant; (b) preparing a binder solution; (c) coating sugar spheres with the drug layering powder, while spraying a binder solution to obtain drug layered pellets; (d) drying the drug layered pellets; (e) optionally, subcoating the drug layered pellets; (f) coating the pellets of (d) or (e) with a pH dependent polymer or a pH independent polymer; and (g) filling the coated pellets into capsules.

In embodiments, a capsule comprises at least two fractions of coated pellets, wherein one fraction is coated with one or more pH independent polymers and another fraction is coated with one or more pH dependent polymers.

In embodiments, a capsule comprises at least two fractions of coated pellets, wherein fractions are independently coated with one or more pH independent polymers or one or more pH dependent polymers.

Equipment suitable for processing the pharmaceutical compositions of the present application include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans, tray dryers, fluid bed dryers, rotary cone vacuum dryers, and the like, and multi-mills, fluid energy mills, ball mills, colloid mills, roller mills, hammer mills, and the like, equipped with a suitable screen.

In embodiments, the present application includes packaging for dexlansoprazole compositions maintain stability during storage and transportation. The stabilization of the dexlansoprazole compositions of the present application can be improved by using a package form such as a package suppressing the permeation of oxygen and moisture, a package replaced with an inert gas (e.g., a gas not containing oxygen), a vacuum package, and a package having an enclosed deoxidizer. The stabilization is improved using these package forms, by reducing the amount of oxygen, with which the solid preparation is directly brought in contact. When a deoxidizer is enclosed, the pharmaceutical solid preparation is packaged with an oxygen permeating material, and then an outer packaging may be provided.

In embodiments, a desiccant and/or an oxygen absorbant is included as a component of packaging. A desiccant is a hygroscopic substance that induces or sustains a state of dryness (desiccation) in its local vicinity in a moderately well-sealed container. Commonly encountered pre-packaged desiccants are solids, and work through absorption or adsorption of water, or a combination of the two. Desiccants for specialized purposes may be in forms other than solid, and may work through other principles, such as chemical bonding of water molecules. A pre-packaged desiccant is most commonly used to remove excessive humidity that would normally degrade or even destroy products sensitive to moisture. Non-limiting examples of various desiccants are anhydrous calcium sulfate (e.g., Drierite® products), silica gel, calcium sulfate, calcium chloride, montmorillonite clay, and molecular sieves. Oxygen absorbants such as StabilOx® are useful in minimizing the degradation of active agent due to oxidation.

In embodiments, the present application provides pharmaceutical compositions comprising dexlansoprazole or salts, polymorphs, hydrates, esters, isomers, derivatives, or mixtures thereof, together with one or more pharmaceutically acceptable excipients, wherein at least one sodium salt is present as an excipient.

In embodiments, dexlansoprazole compositions of the present application comprise the use of dexlansoprazole in an amorphous form as the active agent, together with at least one basic compound such as metal compound, together with one or more other excipients.

In embodiments, dexlansoprazole compositions of the present application comprise dexlansoprazole in an amorphous form as the active agent, together with at least one basic compound, such as an alkaline metal compound, and one or more other excipients, wherein the dexlansoprazole base is at least partially converted into a dexlansoprazole salt of an alkaline metal in situ during processing.

In embodiments, the present application provides dexlansoprazole compositions prepared using dexlansoprazole base or a salt thereof, or mixtures thereof, having particle size distributions with D₅₀ about 1 μm to about 500 μm.

In embodiments, the present application provides dexlansoprazole compositions prepared using dexlansoprazole base or a salt thereof, or mixtures thereof, having particle size distributions with D₅₀ about 1 μm to about 500 μm, and D₉₀ about 1 μm to about 1000 μm.

The “D” values are used to describe particle size distributions. Dₓ is the percentage (x) of particles in a powder having maximum sizes that do not exceed a specified value. For example, a D₅₀ value of 5 μm describes a particle size distribution where 50 percent of the particles analyzed have sizes of 5 μm or less.

In embodiments, the present application provides stable pharmaceutical compositions comprising dexlansoprazole or its pharmaceutically acceptable salts, which are substantially free of drug degradation impurities. The compositions of the present application may contain any one or more of impurities A, B, C, D, and E, described below, or any other drug-related impurities, in amounts such that the impurities do not substantially adversely affect the safety of the composition.

In embodiments, the application provides stable compositions comprising dexlansoprazole or its pharmaceutically acceptable salts, wherein the levels of one or more of impurities A, B, C, D, or E, described herein, are less than about 5%, or less than about 1%, of the label dexlansoprazole content.

In embodiments, the application provides stable compositions comprising dexlansoprazole or its pharmaceutically acceptable salts, wherein levels of the adduct impurity are less than about 5%, or less than about 1%, of the label dexlansoprazole content.

In embodiments, the application relates to stable compositions wherein total drug-related impurities are less than about 7%, or less than about 3%, of the label dexlansoprazole content.

Names and structures for the above-mentioned impurities are shown below.

### Impurity A: 2-mercapto-1H-benzimidazole

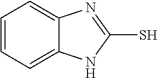

### Impurity B (N-Oxide): 2(((1H-benzimidazole-2-yl)sulfinyl)methyl)-3-methyl-4-((2,2,2-trifluoroethoxy)-pyridine-1-oxide

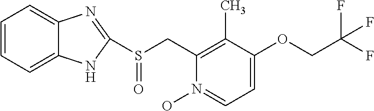

### Impurity C (Nitrosulphoxide): (R)-(+)-2-(4-nitro-3-methyl-pyridin-2-ylmethanesulfinyl)-1H-benzimidazole

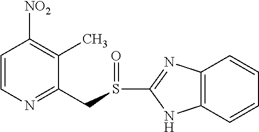

### Impurity D (Sulphone): (2-(((3-Methyl-4-(2,2,2-trifluoroethoxy)-2-pyridyl)methyl-4-sulfonyl)benzimidazole)

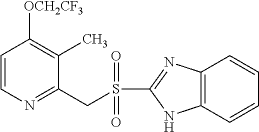

### Impurity E (Sulphide): 3,2-(((3-methyl-4-(2,2,2-trifluoroethoxy)-pyridin-2-yl)methyl)sulfanyl)-1H-benzimadazole

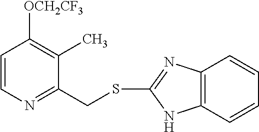

### Adduct Impurity: 1-(1H-benzoimidazol-2-ylsulfanyl)-1-methyl-2-(2,2,2-trifluoroethoxy)-4-a,5,9b-triazaindeno[2,1-a]indene

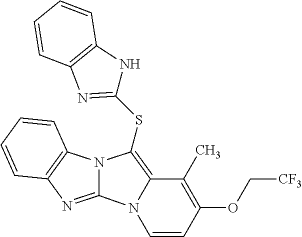

Dexlansoprazole and its impurities, including the above-mentioned impurities, can be analyzed by a high performance liquid chromatography (HPLC) method using a C-18 column (Xterra RP-18,150×4.6 mm, 5 μm), and the following parameters:

Flow rate: 0.8 mL/minute.

Detector wavelength: 285 nm.

Column temperature: 30±2° C.

Run time: 60 minutes.

Mobile phase A: Water.

Mobile phase B: A mixture of acetonitrile, water and triethylamine, in the volume ratio of 160:40:1, respectively.

Gradient elution program:

| | Mobile Phase | Mobile Phase |
|---|---|---|
| Minutes | A (%) | B (%) |
| 0 | 90 | 10 |
| 40 | 20 | 80 |
| 50 | 20 | 80 |
| 51 | 90 | 10 |
| 60 | 90 | 10 |

A 200 ppm test solution is prepared in pH 11.0 borate buffer and a 40 μL sample is injected into the reverse-phase C-18 column of a gradient high-performance liquid chromatography unit. Absorbance is monitored at 285 nm. Borate buffer of pH 11.0 is used as a blank.

For the analysis, a blank is injected, followed by two injections of diluted standard, then a sample. Among the observed peaks are those having the relative response factors and relative retention times (dexlansoprazole=1) approximately as shown below.

| IMPURITY | RRT | RRF |
|---|---|---|
| A | 0.47 | 3.04 |
| B | 0.80 | 1.03 |
| C | 0.81 | 1.17 |
| D | 1.09 | 0.83 |
| E | 1.27 | 1.03 |
| Adduct | 1.56 | 1.77 |

In embodiments, the present application provides processes for producing stable granules, wherein granules are processed in an environment where the relative humidity is not more than about 70%. In embodiments, the present application provides processes for producing stable granules, including drying granules at temperatures of 50±20° C. In embodiments, the present application provides processes for producing stable granules, wherein granules have a water content of 0.5-10% by weight, as determined using a Karl Fischer method. In embodiments, the present application provides processes for producing stable granules, wherein granules have a loss on drying in the range of about 0.02-10%, or about 0.1-5%, by weight.

In embodiments of the present application, the volume ratios of water to organic solvent in the drug loading solution for coating of a substrate are about 100:0 to about 0:100.

In embodiments, the present application provides stable pharmaceutical compositions comprising dexlansoprazole or its pharmaceutically acceptable salts, or mixtures thereof, and at least one pharmaceutically acceptable excipient, wherein pH values of the compositions are less than about 14 when the composition is dispersed in water.

The dosage forms can be subjected to in-vitro dissolution testing according to Test 711 “Dissolution” in United States Pharmacopeia 29, United States Pharmacopeial Convention, Inc., Rockville, Md., 2005, to determine the rate at which the active substance is released from the dosage forms, and content of active substance can be determined in dissolution media using techniques such as high performance liquid chromatography (HPLC).

In embodiments, the present application provides tamper-resistant packages and/or thermo-insulated packages for pharmaceutical dosage forms of dexlansoprazole or its pharmaceutically acceptable salts. The compositions may be packed into blisters, strips, or containers such as plastic, glass, or metal containers. The package or packaging material optionally may contain one or more oxygen absorbents or desiccants.

In embodiments, compositions of the present application can be packaged into thermo-insulated packs wherein aluminum blisters or HDPE bottle containers containing the composition are packed into a suitable thermo-insulated device such as a Thermocol or an extended polystyrene pack. In further embodiments, the container containing the composition is double-walled wherein a hollow space between the walls is filled with air that acts as an insulator. In further embodiments, either one or both of the top and base foils used to make blisters containing the composition contains one or more layers of a desiccant.

In aspects, the present application provides methods of preventing or treating diseases or disorders using compositions of the present application comprising at least one substituted benzimidazole derivative or its salts, or mixtures thereof.

Embodiments of the present application provide methods for producing a gastric acid secretion inhibitory effect to a subject in need thereof, by administering an effective amount of the pharmaceutical compositions comprising at least one substituted benzimidazole derivative or its salts or mixtures thereof.

In an aspect, compositions of the present application exhibit appreciable chemical storage stability, during the shelf life of the product.

In an aspect, the application provides processes for producing stable compositions of dexlansoprazole, embodiments comprising:

a) combining amorphous dexlansoprazole, at least one metal compound, and one or more pharmaceutically acceptable excipients with a solvent to obtain a dexlansoprazole solution;

b) granulating an excipient or mixture of excipients with the material of a);

c) drying the granules followed by optional addition of one or more extra-granular excipients; and

d) formulating the material of c) into a dosage form.

In embodiments, the present application provides multiple unit dosage forms, comprising: (a) pharmacologically inert cores; (b) a drug layer over the cores comprising dexlansoprazole or its salts, or mixtures thereof, and one or more pharmaceutically acceptable excipients; (c) an intermediate layer over the drug layer; and (d) an enteric layer over the intermediate layer.

In embodiments, the present application provides multiple unit dosage forms, comprising: (a) pharmacologically inert cores; (b) a drug layer over the cores, comprising dexlansoprazole base or its sodium salt, or mixtures thereof, and one or more pharmaceutically acceptable excipients; (c) an intermediate layer over the drug layer; and (d) an enteric layer over the intermediate layer.

In embodiments, the present application provides processes for preparing pharmaceutical compositions, embodiments comprising: (a) seal coating inert sugar spheres with a polymer solution; (b) applying a dexlansoprazole drug coating layer, comprising sodium hydroxide, onto the spheres obtained in (a); (c) applying an intermediate layer onto the drug coated spheres obtained in (b); and (d) applying an enteric coating layer onto the intermediate layer coated spheres obtained in (c); wherein a dexlansoprazole sodium salt is formed in situ during the process.

In embodiments, the active agent dexlansoprazole is contained in the compositions as a free base or salt form during manufacture or storage, in an amorphous form, a crystalline form, or mixtures thereof.

In embodiments, compositions of the present application are in the form of multiple unit capsules. Compositions of the present application can be prepared using the techniques described herein, as well as other methods known to those having skill in the art.

The final dosage form can be an enteric coated tablet or capsule, or in the case of enteric coated pellets or granules, the pellets or granules can be dispensed in hard gelatin capsules or sachets. The final dosage form may further be coated with an additional layer containing pigments, colorants, and/or moisture-protecting agents.

Pharmaceutical preparations according to the present application are especially advantageous in prevention and treatment of gastric acid-related disorders in man and other mammals, including, for example, gastroesophageal reflux disease, gastritis, gastric ulcers, duodenal ulcers, etc. They are typically administered one to several times per day. A typical daily dose of the active compound will depend on various factors, such as, for example, the individual requirement of a patient, the route of administration and the disease. In general, oral and parenteral dosages will be in the range of 2.5 to 500 mg per day of active substance.

Certain specific aspects and embodiments of the application will be explained in more detail with reference to the following examples, being provided only for purposes of illustration, and it is to be understood that the present application is not deemed to be limited thereto.

### Examples 1-2

### Dexlansoprazole 60 Mg and 30 Mg Capsules

| | mg per Capsule | |
|---|---|---|
| Ingredient | 1 | 2 |

Seal Coating

| | | |
|---|---|---|
| Sugar spheres (25-30 mesh) | 86 | 43 |
| Hydroxypropyl methylcellulose, 5 cps | 4 | 2 |
| Methylene chloride* | q.s. | q.s. |
| Methanol* | q.s. | q.s. |

Drug Loading

| | | |
|---|---|---|
| Dexlansoprazole (amorphous) | 60 | 30 |
| Sodium hydroxide | 1.63 | 0.82 |
| Polyvinylpyrrollidone K30 | 20 | 10 |
| Talc | 10 | 5 |
| Water* | q.s. | q.s. |
| Methanol* | q.s. | q.s. |

Subcoating

| | | |
|---|---|---|
| Hydroxypropyl methylcellulose, 5 cps | 12.87 | 6.43 |
| Talc | 5.15 | 2.57 |
| Titanium dioxide | 7.72 | 3.86 |
| Water* | q.s. | q.s. |

Enteric Coating
I. Delayed Release Portion

| | | |
|---|---|---|
| Methacrylic acid copolymer type C, 30% aqueous | 15.8 | 7.9 |
| dispersion (Eudragit ® L100-55) | | |
| Triethyl citrate | 1.7 | 0.85 |
| Talc | 7.9 | 3.95 |
| Isopropyl alcohol* | q.s. | q.s. |
| Water* | q.s. | q.s. |

II. Extended Release Portion

| | | |
|---|---|---|
| Methacrylic acid copolymer type B (Eudragit ® | 53.01 | 26.505 |
| S 100) | | |
| Methacrylic acid copolymer type A (Eudragit ® | 17.68 | 8.84 |
| L 100) | | |
| Talc | 21.2 | 10.6 |
| Triethyl citrate | 6.83 | 3.415 |
| Isopropyl alcohol* | 92 parts | 92 parts |
| Water* | 8 parts | 8 parts |

*Evaporates during processing.

Manufacturing Process:

### A. Seal Coating

1. Hydroxypropyl methylcellulose (5 cps) is dissolved in a mixture of methanol and methylene chloride.

2. The solution is sprayed onto sugar spheres using a fluid bed processor (FBP) to achieve a weight gain of 5%.

### B. Drug Loading

1. Sodium hydroxide is dissolved in water, then dexlansoprazole is added and stirred.

2. Polyvinylpyrrollidone K30 is dissolved in methanol and added to the above solution. The volume ratio of water to methanol in the mixture is 25:75.

3. Talc is dispersed in the above solution, maintained under stirring.

4. The drug solution is sprayed onto seal coated sugar spheres.

5. The particles are dried at 40° C. until loss on drying (LOD) is less than 2% w/w, measured at 60° C.

### C. Subcoating

1. Hydroxypropyl methylcellulose (5 cps) is dissolved in water.

2. Talc and titanium dioxide are homogenized with water for 20-30 minutes.

3. The dispersion of step 2 is added to the polymer solution with stirring.

4. The dispersion of step 3 is coated onto the drug loaded sugar spheres from B, using a fluid bed processor.

### D. Enteric Coating of Delayed Release Portion

1. Eudragit® L100-55 is dissolved in isopropyl alcohol.

2. Water and triethyl citrate are added to the Eudragit solution with stirring.

3. Talc is added to the solution of step 2, with stirring.

4. The dispersion of step 2 is sprayed onto 25% of the subcoated pellets, using a FBP with bottom spray, to achieve a weight gain of 15±5%.

5. Enteric coated pellets are dried in the FBP until LOD of the pellets is 1-3% w/w, measured at 60° C. using a halogen moisture balance.

6. The pellets are cured in the FBP for 2 hours at 40° C.

### E. Enteric Coating of Extended Release Portion

1. Triethyl citrate is dissolved in isopropyl alcohol.

2. Eudragit® S100 and Eudragit® L100 are dissolved in the above solution with stirring.

3. Water is added with stirring to form a clear solution.

4. Talc is added to solution of step 3, and the dispersion is maintained under continuous stirring until coating is completed.

6. The dispersion is sprayed onto 75% of the subcoated pellets using a FBP with bottom spray, to achieve a weight gain of 55±5%.

7. Enteric coated pellets are dried in the FBP until LOD is 1-3% w/w, measured at 60° C. using a halogen moisture balance.

8. The pellets are cured in the FBP for 2 hours at 40° C.

### F. Encapsulation

1. Enteric coated pellets (delayed release+extended release) are filled into capsules: size 1 for Example 1; and size 3 for Example 2.

The capsules of Example 1 are packaged in a closed HDPE container with 3 g of molecular sieve as a desiccant, and stored under accelerated stability testing conditions of 40° C. and 75% RH for 3 months. The samples are analyzed for impurities and drug polymorphic form, and results are shown below, where values are percentages of the label drug content.

| Parameter | Example 1 |
|---|---|
| Impurity A | 0.18 |
| Impurity B | ND |
| Impurity C | ND |
| Impurity D | 0.29 |
| Impurity E | 0.21 |
| Adduct impurity | 0.23 |
| Total impurities | 1.89 |
| Polymorphic form | Amorphous |

ND = Not detected.

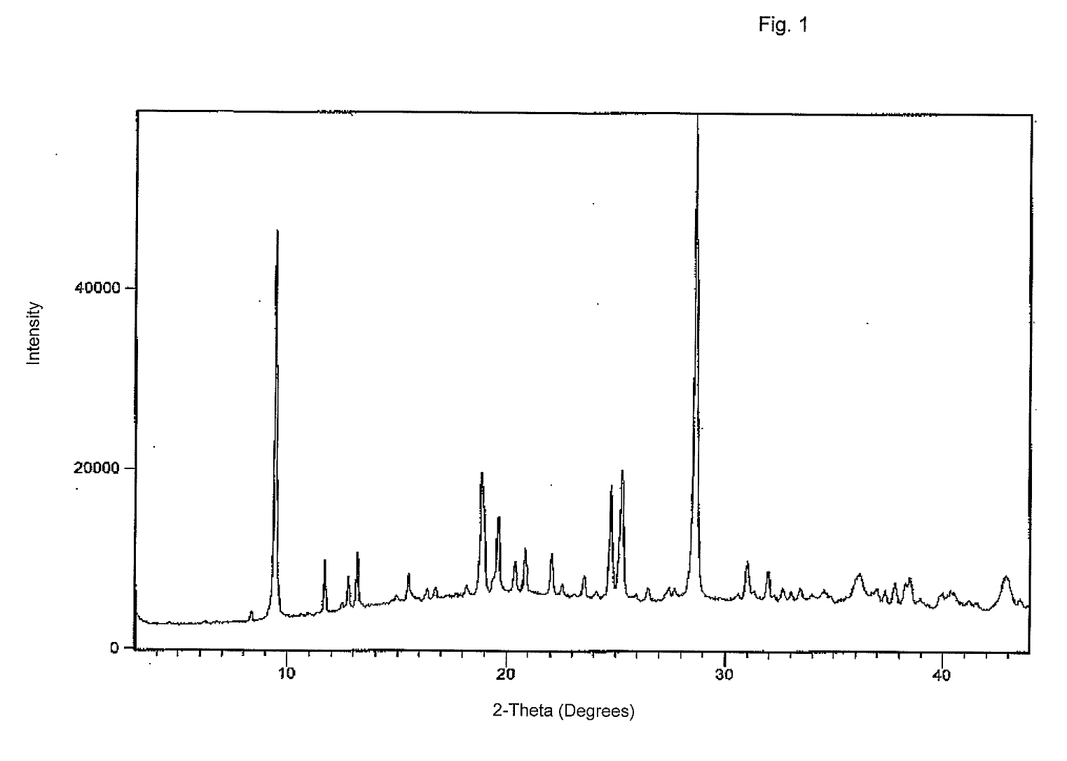

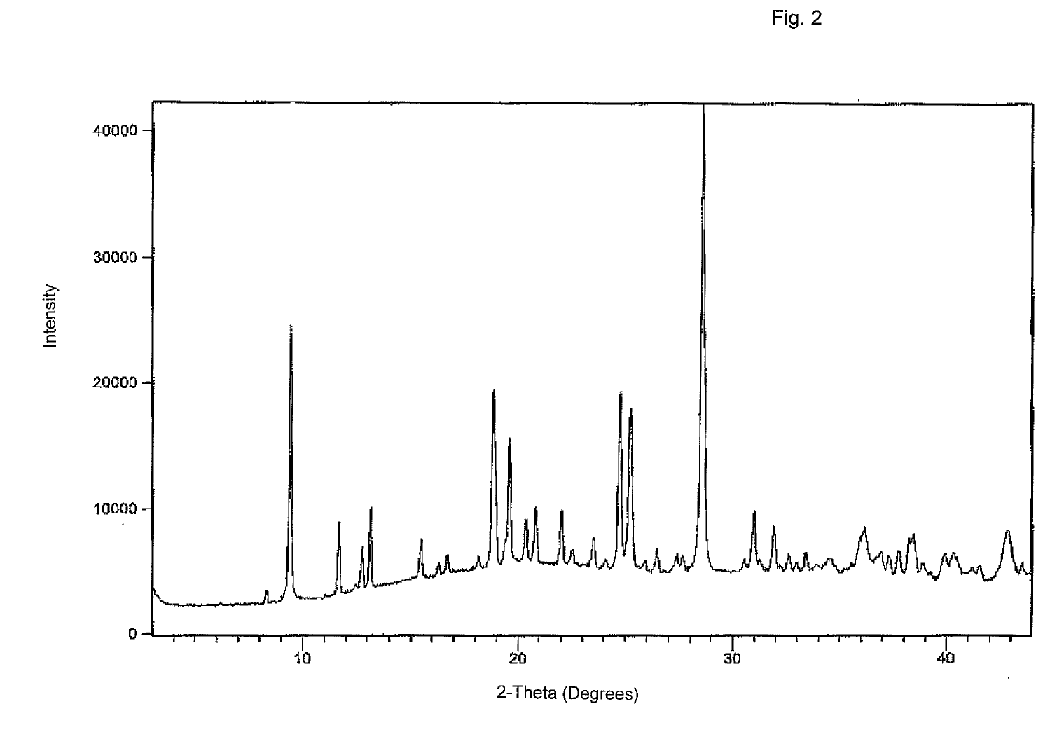

FIG. 1 represents the XRPD pattern of Example 1 capsule contents before storage, and FIG. 2 represents the pattern of the capsule contents after storage. Dexlansoprazole retains its polymorphic form during the storage.

### Examples 3-5

### Dexlansoprazole 60 Mg Delayed Release Capsules

| | mg per Capsule | | |
|---|---|---|---|
| Ingredient | 3 | 4 | 5 |

Seal Coating

| | | | |
|---|---|---|---|
| Sugar spheres (25-30 mesh) | 86 | 86 | 86 |
| Hydroxypropyl methylcellulose, 5 cps | 4 | 4 | 4 |
| Methylene chloride* | q.s. | q.s. | q.s. |
| Methanol* | q.s. | q.s. | q.s. |

Drug Loading

| | | | |
|---|---|---|---|
| Dexlansoprazole (amorphous) | 60 | 60 | 60 |
| Sodium hydroxide | 6.5 | 3.25 | 1.625 |
| Polyvinylpyrrollidone K30 | 20 | 20 | — |
| Hydroxypropyl methylcellulose E5 | — | — | 15 |
| Talc | 10 | 10 | 10 |
| Water* | q.s. | q.s. | q.s. |
| Methanol* | q.s. | q.s. | q.s. |

Subcoating

| | | | |
|---|---|---|---|
| Hydroxypropyl methylcellulose, 5 cps | 12.87 | 12.87 | 12.87 |
| Talc | 5.15 | 5.15 | 5.15 |
| Titanium dioxide | 7.72 | 7.72 | 7.72 |
| Water* | q.s. | q.s. | q.s. |

Enteric Coating
I. Delayed Release Portion

| | | | |
|---|---|---|---|
| Methacrylic acid copolymer type C, 30% | 15.8 | 15.8 | 15.8 |
| aqueous dispersion (Eudragit ® L100-55) | | | |
| Triethyl citrate | 1.7 | 1.7 | 1.7 |
| Talc | 7.9 | 7.9 | 7.9 |
| Isopropyl alcohol* | q.s. | q.s. | q.s. |
| Water* | q.s. | q.s. | q.s. |

II. Extended Release Portion

| | | | |
|---|---|---|---|
| Methacrylic acid copolymer type B | 53.01 | 53.01 | 53.01 |
| (Eudragit ® S 100) | | | |
| Methacrylic acid copolymer type A | 17.68 | 17.68 | 17.68 |
| (Eudragit ® L 100) | | | |
| Talc | 21.2 | 21.2 | 21.2 |
| Triethyl citrate | 6.83 | 6.83 | 6.83 |
| Isopropyl alcohol* | q.s. | q.s. | q.s. |
| Water* | q.s. | q.s. | q.s. |

*Evaporates during processing.

Manufacturing process is similar to the process described for Examples 1 and 2. For drug loading, the volume ratios of water to methanol are: 50:50 for Example 3; 33.3:66.7 for Example 4; and 90:10 for Example 5. For enteric coating, the volume ratio of isopropyl alcohol to water is 92:8. Delayed release and extended release pellets are filled into size 1 capsules.

### Examples 6-8

### Dexlansoprazole 60 Mg Delayed Release Capsules

| | mg per Capsule | | |
|---|---|---|---|
| Ingredient | 6 | 7 | 8 |

Seal Coating

| | | | |
|---|---|---|---|
| Sugar spheres (25-30 mesh) | 86 | 86 | 86 |
| Hydroxypropyl methylcellulose, 5 cps | 4 | 4 | 4 |
| Methylene chloride* | q.s. | q.s. | q.s. |
| Methanol* | q.s. | q.s. | q.s. |

Drug Loading

| | | | |
|---|---|---|---|
| Dexlansoprazole (amorphous) | 60 | 60 | 60 |
| Sodium hydrogen carbonate | 6.8 | 3.41 | 1.72 |
| Potassium hydroxide | 6.8 | 3.41 | 1.72 |
| Polyvinylpyrrollidone K30 | 20 | 20 | 20 |
| Talc | 10 | 10 | 10 |
| Water* | q.s. | q.s. | q.s. |
| Methanol* | q.s. | q.s. | q.s. |

Subcoating

| | | | |
|---|---|---|---|
| Hydroxypropyl methylcellulose, 5 cps | 12.87 | 12.87 | 12.87 |
| Talc | 5.15 | 5.15 | 5.15 |
| Titanium dioxide | 7.72 | 7.72 | 7.72 |
| Water* | q.s. | q.s. | q.s. |

Enteric Coating
I. Delayed Release Portion

| | | | |
|---|---|---|---|
| Methacrylic acid copolymer type C, 30% | 15.8 | 15.8 | 15.8 |
| aqueous dispersion (Eudragit ® L100-55) | | | |
| Triethyl citrate | 1.7 | 1.7 | 1.7 |
| Talc | 7.9 | 7.9 | 7.9 |
| Isopropyl alcohol* | q.s. | q.s. | q.s. |
| Water* | q.s. | q.s. | q.s. |

II. Extended Release Portion

| | | | |
|---|---|---|---|
| Methacrylic acid copolymer type B (Eudragit ® S | 53.01 | 53.01 | 53.01 |
| 100) | | | |
| Methacrylic acid copolymer type A (Eudragit ® L | 17.68 | 17.68 | 17.68 |
| 100) | | | |
| Talc | 21.2 | 21.2 | 21.2 |
| Triethyl citrate | 6.83 | 6.83 | 6.83 |
| Isopropyl alcohol* | q.s. | q.s. | q.s. |
| Water* | q.s. | q.s. | q.s. |

*Evaporates during processing.

Manufacturing process is similar to the process described for Examples 1 and 2. For drug loading, the volume ratios of water to methanol are: 20:80 for Example 6; 16.7:83.3 for Example 7; and 14.3:85.7 for Example 8. For enteric coating, the volume ratio of isopropyl alcohol to water is 92:8. Delayed release and extended release pellets are filled into size 1 capsules.

### Examples 9-10

### Dexlansoprazole 30 Mg and 15 Mg Gastro-Resistant Capsules

| | mg per Capsule | |
|---|---|---|
| Ingredient | 9 | 10 |

Seal Coating

| | | |
|---|---|---|
| Sugar spheres | 23.81 | 11.91 |
| Hypromellose USP 2906 (Methocel ™ E5 Premium | 0.92 | 0.46 |
| LV) | | |
| Magnesium stearate | 0.18 | 0.09 |
| Diethyl phthalate | 0.09 | 0.05 |
| Isopropyl alcohol* | q.s. | q.s. |
| Dichloromethane* | q.s. | q.s. |

Drug Coating

| | | |
|---|---|---|
| Dexlansoprazole | 30 | 15 |
| Potassium hydroxide | 6.5 | 3.25 |
| Hydroxypropyl cellulose (Klucel ™ LF) | 15 | 7.5 |
| Meglumine | 5 | 2.5 |
| Amorphous silica (Syloid ™ 244FP) | 1 | 0.5 |
| Methanol* | q.s. | q.s. |

Subcoating

| | | |
|---|---|---|
| Hypromellose 2906 (Methocel E5 Premium LV) | 21.35 | 10.68 |
| Magnesium stearate | 4.27 | 2.14 |
| Diethyl phthalate | 2.13 | 1.07 |
| Isopropyl alcohol* | q.s. | q.s. |
| Dichloromethane* | q.s. | q.s. |

Enteric Coating

| | | |
|---|---|---|
| Poly(methacrylic acid, ethyl acrylate) 1:1 (Eudragit | 60.01 | 30.06 |
| L100 55) | | |
| Talc | 30.06 | 15.03 |
| Diethyl phthalate | 6.01 | 3.01 |
| Isopropyl alcohol and methanol* | q.s. | q.s. |

*Evaporates during processing.

Manufacturing Process:

### A. Seal Coating

1. Hypromellose is dispersed in isopropyl alcohol (IPA) with stirring, followed by addition of dichloromethane with stirring.

2. Magnesium stearate is homogenized with IPA for 30 minutes and then added to the dispersion of step 1.

3. Diethyl phthalate is dissolved in dichloromethane, added to the dispersion of step 2, and stirred for 15 minutes.

4. The dispersion is filtered through a #80 mesh sieve.

5. Sugar spheres are loaded into a fluid bed coater (FBC) and warmed for 10 minutes to 40° C.

6. The dispersion prepared in step 4 is sprayed onto sugar spheres to achieve a weight gain of 5%.

7. Coated pellets are dried in the FBC for 20 minutes at 40° C.

### B. Drug Coating

1. Potassium hydroxide is dissolved in methanol with stirring.

2. Dexlansoprazole sodium amorphous (equivalent to the stated amount of dexlansoprazole) is dissolved in methanol with stirring.

3. Klucel LF, meglumine, and Syloid 244 FP are dispersed in methanol with stirring.

4. The solution of step 1 is added slowly to step 2 with stirring until complete precipitation occurs. Stirring is continued for 1 hour.

5. The dispersion of step 3 is added to the dispersion of step 4 and stirring is continued for 30 minutes, then the dispersion is passed through a #80 mesh sieve.

6. Seal coated pellets from A are loaded into a FBC and the dispersion of step 6 is sprayed onto the pellets at 30-35° C.

7. After spraying, the pellets are dried at 32° C. for 15-30 minutes.

### C. Subcoatinq

1. Hypromellose is dispersed in IPA with stirring, followed by addition of dichloromethane with stirring.

2. Magnesium stearate is homogenized with IPA for 30 minutes and then added to the dispersion of step 1.

3. Diethyl phthalate is dissolved in dichloromethane, added to the dispersion step 2, stirred for 15 minutes, and filtered through a #80 mesh sieve.

4. Drug coated pellets from B are loaded into a FBC and coated with the dispersion of step 3 at 30-35° C.

5. Coated pellets are dried at 32° C. for 15-30 minutes.

### D. Enteric Coating

1. Eudragit L100 55 is dissolved in IPA with stirring.

2. Talc is homogenized in IPA for 30 minutes and then added to the solution of step 1.

3. Diethyl phthalate is dissolved in IPA, added to the dispersion of step 2 and stirred for 15 minutes, then the dispersion is filtered through a #80 mesh sieve.

4. Subcoated pellets from C are loaded into a FBC, the enteric coating dispersion of step 3 is sprayed onto the pellets at 30-35° C., and the pellets are dried.

### E. Filling

Enteric coated pellets are filled into size 1 capsules.

### Example 11

### Dexlansoprazole 30 Mg Tablets

| | |
|---|---|
| Ingredient | mg per Tablet |

Seal Coating

| | |
|---|---|
| Sugar spheres | 23.81 |
| Hypromellose 2906 (Methocel E5 Premium LV) | 0.92 |
| Magnesium stearate | 0.18 |
| Diethyl phthalate | 0.09 |
| Isopropyl alcohol* | q.s. |
| Dichloromethane* | q.s. |

Drug coating

| | |
|---|---|
| Dexlansoprazole | 30 |
| Sodium hydroxide | 3.5 |
| Magnesium carbonate | 3 |
| Hydroxypropyl cellulose (Klucel LF) | 15 |
| Meglumine | 5 |
| Syloid 244 FP | 1 |
| Methanol* | q.s. |

Subcoating

| | |
|---|---|
| Hypromellose 2906 (Methocel E5 Premium LV) | 21.35 |
| Magnesium stearate | 4.27 |
| Diethyl phthalate | 2.13 |
| Isopropyl alcohol* | q.s. |
| Dichloromethane* | q.s. |

Enteric Coating

| | |
|---|---|
| Poly(methacrylic acid, ethyl acrylate) 1:1 (Eudragit | 56.37 |
| L100 55) | |
| Talc | 28.18 |
| Diethyl phthalate | 5.64 |
| Isopropyl alcohol* | q.s. |

Overcoating

| | |
|---|---|
| PEG 6000 | 31.57 |
| Methanol* | q.s. |
| Methylene chloride* | q.s. |

F. Blending, Lubrication, and Compression

| | |
|---|---|
| Mannitol (Pearlitol ™ SD 200) | 375.54 |
| Microcrystalline cellulose (Avicel PH 200) | 160.95 |
| Crospovidone (Polyplasdone ™ XL) | 45 |
| Amorphous silica (Syloid 244FP) | 9 |
| PEG 600 | 22.5 |
| Copovidone (Kollidon ™ VA 64) | 36 |
| Magnesium stearate | 9 |

Final Coating

| | |
|---|---|
| Opadry ™ AMB OY-B29000** | 27 |
| Water* | q.s. |

*Evaporates during processing.
**Opadry AMB OY-B29000 is a product of Colorcon and contains partially hydrolyzed polyvinyl alcohol, talc, soya lecithin, and xanthan gum.

Manufacturing Process:

Enteric coated pellets are prepared from ingredients listed in A-D, in a manner similar to that described for Examples 9 and 10. The enteric coated pellets are then overcoated using a PEG 6000 solution, and dried. The overcoated pellets are blended with mannitol, microcrystalline cellulose, crospovidone, silica, PEG 600, and copovidone for 15 minutes. The mixture is blended with magnesium stearate and compressed into tablets. Tablets are coated with the Opadry product in water, and dried.

1. A pharmaceutical formulation comprising: (a) a pharmacologically inert core; (b) a drug layer over the core comprising dexlansoprazole, or a salt thereof, and one or more pharmaceutically acceptable excipients; (c) an intermediate layer over the drug layer; and (d) an enteric layer over the intermediate layer.

2. The pharmaceutical formulation of claim 1, wherein dexlansoprazole or a salt thereof is amorphous.

3. The pharmaceutical formulation of claim 1, wherein (b) further comprises a metal compound.

4. The pharmaceutical formulation of claim 2, wherein a metal compound comprises sodium hydroxide, sodium chloride, sodium carbonate, sodium hydrogen carbonate, or any combination thereof.

5. The pharmaceutical formulation of claim 3, wherein a dexlansoprazole metal salt is formed in (b).

6. The pharmaceutical formulation of claim 4, wherein a dexlansoprazole metal salt is formed in (b).

7. The pharmaceutical formulation of claim 1, wherein a dexlansoprazole salt is dexlansoprazole sodium.

8. The pharmaceutical formulation of claim 1, in the form of granules, pellets, spherules, spheroids, micro-tablets, or mini-tablets.

9. The pharmaceutical formulation of claim 1, made into a tablet or capsule unit dosage form.

10. A pharmaceutical formulation comprising: (a) a pharmacologically inert core; (b) a drug layer over the core comprising amorphous dexlansoprazole, or a salt thereof, a metal compound, and one or more pharmaceutically acceptable excipients; (c) an intermediate layer over the drug layer; and (d) an enteric layer over the intermediate layer.

11. The pharmaceutical formulation of claim 10, wherein (b) comprises dexlansoprazole and a metal compound, and a dexlansoprazole salt is formed in situ by their reaction.

12. The pharmaceutical formulation of claim 11, wherein a metal compound comprises sodium hydroxide, sodium chloride, sodium carbonate, sodium hydrogen carbonate, or any combination thereof.

13. A process for preparing a pharmaceutical formulation, comprising:
(a) combining dexlansoprazole, a metal compound, and one or more pharmaceutically acceptable excipients with a solvent, to obtain a dexlansoprazole solution;
(b) applying the dexlansoprazole solution obtained in (a) onto pharmacologically inert cores;
(c) applying an intermediate layer onto drug coated cores obtained in (b); and
(d) applying an enteric coating layer onto intermediate layer coated cores obtained in (c);
wherein a dexlansoprazole metal salt is formed in situ during the process.

14. The process of claim 13, further comprising:
(e) mixing enteric coated cores with one or more pharmaceutically acceptable excipients; and
(f) making the composition thus obtained into a tablet or capsule unit dosage form.

15. The process of claim 13, wherein dexlansoprazole is amorphous.

16. The process of claim 13, wherein a metal compound comprises sodium hydroxide, sodium chloride, sodium carbonate, sodium hydrogen carbonate, or any combination thereof.

17. The process of claim 13, wherein an in situ formed salt is a sodium salt.
